(19) United States
(12) Patent Application Publication
(10) Pub. No.: US 2005/0009170 A1
(43) Pub. Date: Jan. 13, 2005

(54) Preparation of metal nanoparticles in plants

(75) Inventors: Jorge L. Gardea-Torresdey, El Paso, TX (US); Jason G. Parsons, Calgary (CA); Jose R. Peralta-Videa, El Paso, TX (US); Eduardo Gomez, El Paso, TX (US); Miguel Jose-Yacaman, Lakeway, TX (US)

Correspondence Address: FULBRIGHT & JAWORSKI L. L. P., SUITE 2400, 600 CONGRESS AVENUE, AUSTIN, TX, 78701-3271, US

(73) Assignee: The University of Texas System

(21) Appl. No.: 10/732,927

(22) Filed: Dec. 10, 2003

Related U.S. Application Data
(60) Provisional application No. 60/432,160, filed on Dec. 10, 2002.

(51) Int. Cl. A01H 1/00; C12N 15/82

(52) U.S. Cl. 435/262; 800/278

(57) ABSTRACT

The present invention relates to the use of plants for the production of metal nanoparticles. In particular, precious metals such as gold, silver and platinum can be produced using plants such as alfalfa. The nanoparticles have a wide variety of uses

This application claims the benefit of priority to U.S. Provisional Ser. No. 60/432,160, filed Dec. 10, 2002, the entire contents of which are hereby incorporated by reference.

The government may have rights in the present invention pursuant to grants from: the National Institutes of Health (#S06GM8012-30), Office of Exploratory Research of the EPA (#CR-819849-01-4) and the Department of Energy (Cooperative Agreement #DE-FCO4-908L6G158; Clark Atlanta University HBCU/MI Environmental Technology Consortium).

### BACKGROUND OF THE INVENTION

1. Field of the Invention

The present invention relates generally to the fields of botany and cellular biology. More particularly, it concerns the synthesis of metal nanoparticles by plants.

2. Description of Related Art

The field of nanotechnology is one of the most active areas of research in modern materials science. New applications of nanoparticles and nanomaterials are emerging rapidly. Jahn (1999); Dickson (1999); Naiwa (2000). The synthesis of nanoparticles and their self-assembly is a cornerstone of nanotechnology. New methods to manufacture nanoparticles are constantly being studied and developed. Additionally, environmental metal contamination byproducts associated with metal production has become a great concern for environmental reasons. Winterbourn et al. (2000). An example is the processing of valuable metal ores, which provides a route for toxic compounds to enter the environment. A paradigm of this is the current technology to extract gold, in which cyanide is used to release gold from the ore into a solution as gold cyanide, AuCN₂.

Currently, the AuCN₂ is still absorbed onto activated carbon and is subsequently stripped from the carbon followed by electrochemical reduction to gold(0), which is a very expensive process. Recently, it has been shown that several types of inactivated biomasses and living organisms have the ability to remove high concentrations of gold(III) from solution and to reduce gold(III) to gold(0). Gardea-Torresdey et al. (1999); Kuyicek and Volensky (1989); Greene et al. (1986). These studies provide the possibility of an environmentally-friendly method to remediate mining wastes.

Although it is well-known that inactivated biological systems interact with metal ions, the connection between metal ions and biological systems is more in depth. As is well-known, many elements at trace concentrations are essential for plant growth and propagation; however, these same elements at higher concentrations are toxic to some plants. More specifically, it has been shown that many bacteria and plants can actively uptake and bioreduce metal ions from soils and solutions. A well-known example of bioreduction and nanoparticle production is magnetostatic bacteria that can synthesize magnetic nanoparticles. Dickson (1999). Another example of nanoparticle production using inactivated alfalfa biomass has shown that biomass can reduce gold(III) ions in solution to gold(0) nanoparticles. Gardea-Torresdey et al. (1999). The use of inactivated biomass to recover metal ions from solution has been studied extensively. The possibility of using live bacteria for the remediation of metal-contaminated waters has shown the bacterial production of silver-carbon composite materials. Also, the formation of surface trapping of nanoparticles by fungus has been reported. Naiwa (2000); Stephen and McCaughton (1999). These are examples that link biotechnology and material science (nanobiotechnology). Naiwa (2000); Stephen and McCaughton (1999); Mukherjee et al. (2001). It has been well-known for some time that plants can uptake gold from soils as they are used as bioindicators of gold deposits by mining companies. McInnes et al. (1996). Although plants are known to uptake gold from solution and soils, the oxidation state and the form of the gold has never been elucidated.

### SUMMARY OF THE INVENTION

Thus, in accordance with the present invention, there is provided a method of producing a precious metal nanoparticle in a plant comprising (a) selecting a plant growth environment comprising a precious metal source; (b) growing a plant in the plant growth environment; and (c) isolating the precious metal nanoparticle. The precious metal may be gold, silver, or platinum. The plant may be a dicot, such as a dicot of the division Magnoliophyta, in particular alfalfa.

The isolating may comprise isolating a part of the plant, for example, a leaf, a stem, or a root. The method may further comprising disrupting the plant part by physical, chemical or biological methods. Physical methods may comprise pressing, grinding, sonication or burning. Chemical methods may comprise digestion or extraction. Biological methods may comprise enzymatic degradation or microbial degradation. Specific methods for isolation may comprise one or more of chromatography, centrifugation or electrophoresis.

Growing may comprise planting a seed, a sprout of said plant, or said plant. The method may further comprise creating the plant growth environment comprising a precious metal source. The plant growth environment may be soil or liquid. Creating an environment may comprise seeding a solid growth medium with a precious metal. The solid growth medium is soil or agar. Creating a plant growth environment may also comprise mixing a precious metal with a liquid. Specifically, creating a plant growth environment may comprise (i) selecting an desired particle size; and (ii) adjusting the precious metal concentration to produce the desire particle size.

The gold nanoparticles may have one or more of the following characteristics: (i) crystalline; (ii) size of between about 2 nm and about 40 nm; (iii) twinned structure; (iv) icosahedral structure; (v) zero valence. The silver nanoparticles may have one or more of the following characteristics: (i) crystalline; (ii) size of between about 2 nm and 20 nm; (iii) icosahedral structure; (iv) dimeric, multimeric or wired; (v) zero valence.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—XANES of the gold alfalfa roots and shoots and gold-enriched agar samples, gold(0) foil, and the tetrachloroaurate model compound. These data show that the gold present in the alfalfa and agar samples is present as gold(0).

FIG. 2—EXAFS of the gold alfalfa and roots and shoots and gold-enriched agar samples, gold(0) foil, and the tetrachloroaurate model compound. These data show that the nearest neighboring atom in the alfalfa and agar samples is gold.

FIGS. 3A-C—(FIG. 3A) Low-magnification TEM image of the alfalfa shoot showing aggregates of gold nanoparticles. The scale is shown in the image. (FIG. 3B) X-ray EDS analyses confirming that the nanoparticles are constituted by pure Au. (FIG. 3C) Histogram showing the Au nanoparticle size distribution in a typical alfalfa sample.

FIGS. 4A-C—HRTEM images of three different nanoparticles. The inset in the upper left corner of the micrograph corresponds to the FFT. (FIG. 4A) and (FIG. 4B) show different crystalline arrangements. The FFT confirms that the structures are twinned. (FIG. 4C) shows a very small icosahedron tilted 18° from the 3-fold orientation.

FIG. 5—Image showing the coalescence of nanoparticles inside the alfalfa plant. Nanoparticles show atomic resolution or at least fringes indicating an orientation relationship between them.

FIG. 6—XANES of the silver alfalfa roots and shoots; silver agar sample, Ag(0) foil, and the silver nitrate model compound. The data show that the silver present in the alfalfa plant and agar samples is present as Ag(0).

FIG. 7—EXAFS of the silver alfalfa roots and shoots, silver agar sample, Ag(0) foil, and the silver nitrate model compound. The data show the interatomic distances of Ag atom and nearest neighboring atom in the alfalfa and agar samples.

FIGS. 8A-B. (FIG. 8A) Low-magnification TEM image of the alfalfa shoot showing a unidimensional array of silver nanoparticles. The scale is shown in the image. (FIG. 8B) X-ray EDS analysis confirmed that the nanoparticles are constituted by Ag.

FIGS. 9A-B—Dark field TEM images obtained using the Z-contrast high angle annular dark field detector (HAADF). (FIGS. 9A and 9B) Silver nanoparticles shown in a crystalline state as very bright spots being connected, in some cases, by non-crystalline silver nanoparticles or atoms. The scale is shown in the image.

FIGS. 10A-B—HRTEM images of two different nanoparticles being in a crystalline state. (FIG. 10A) Shows one of the smallest silver nanoparticles found, ranging from 2 to 3 nanometers in size and possessing an icosahedral shape. (FIG. 10B) Shows coalescence of silver nanoparticles inside alfalfa plants. The scale is shown in the image.

FIG. 11—Effect of Au on alfalfa elongation.

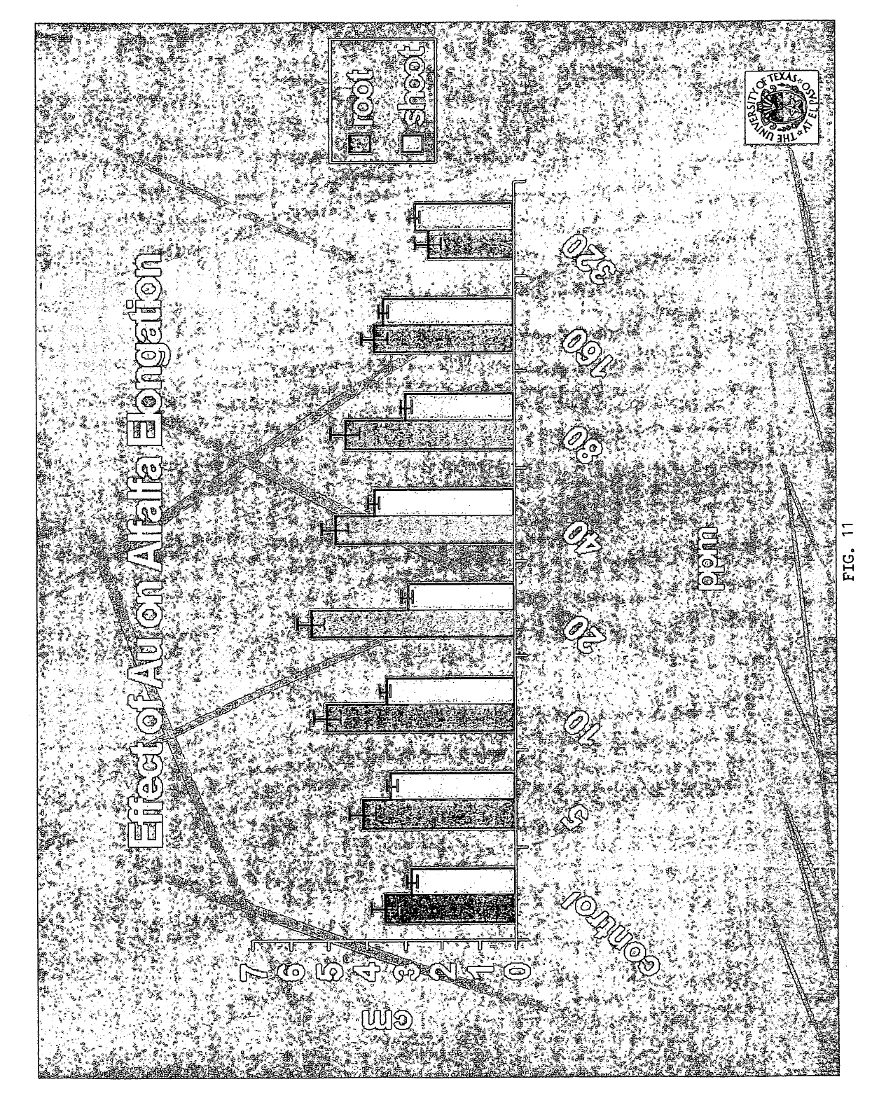

FIG. 12—Au uptake by Alfalfa root.

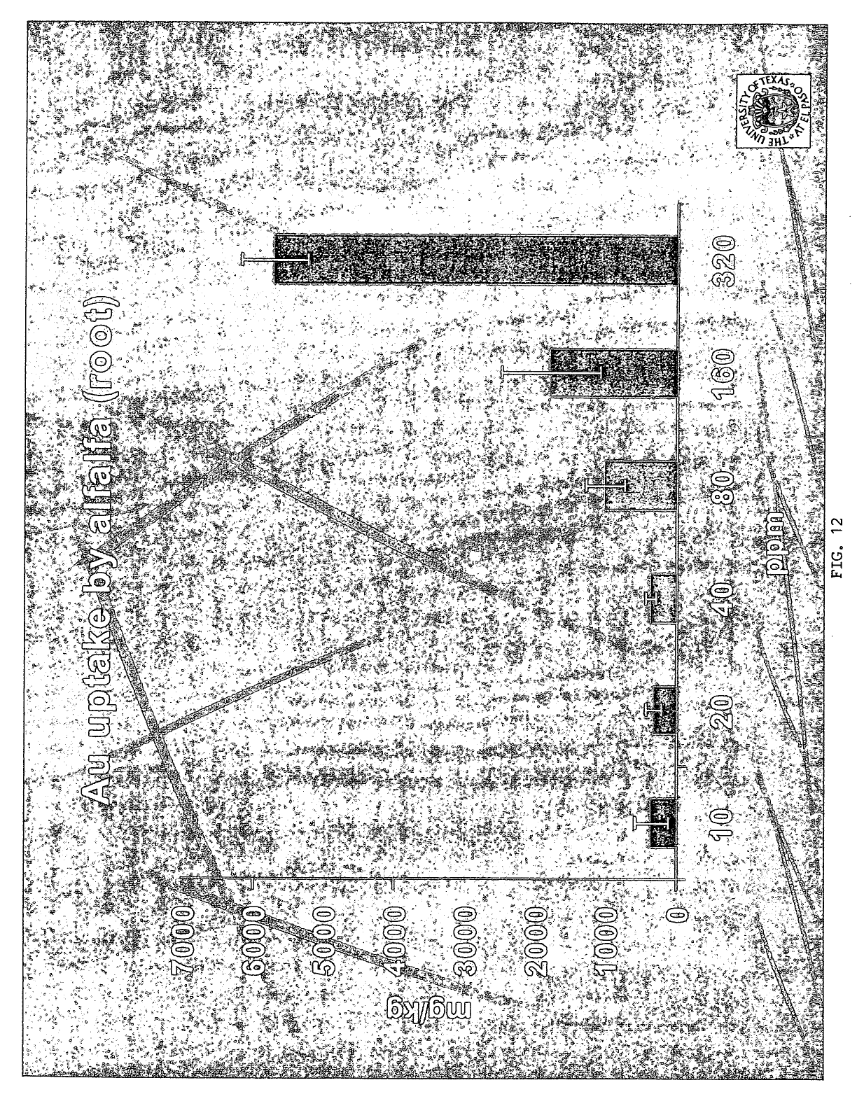

FIG. 13—Au uptake by Alfalfa shoot.

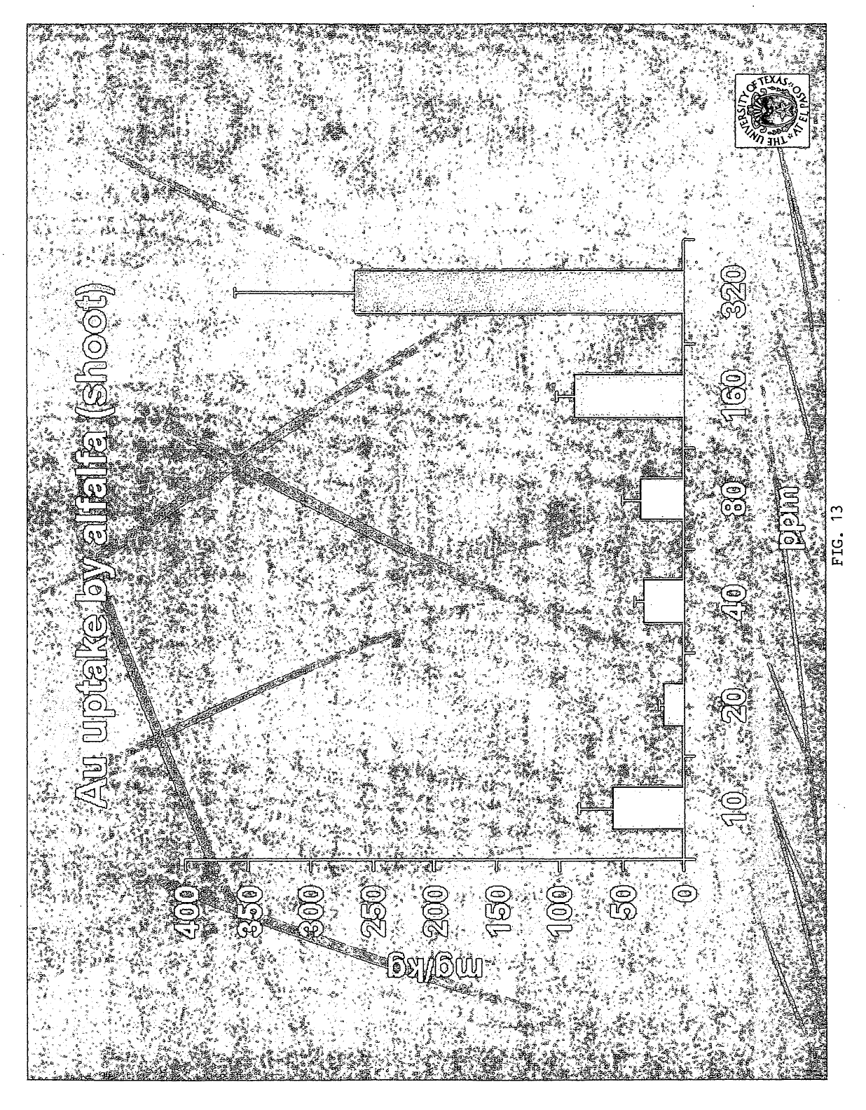

FIG. 14—Effect of Ag on alfalfa elongation.

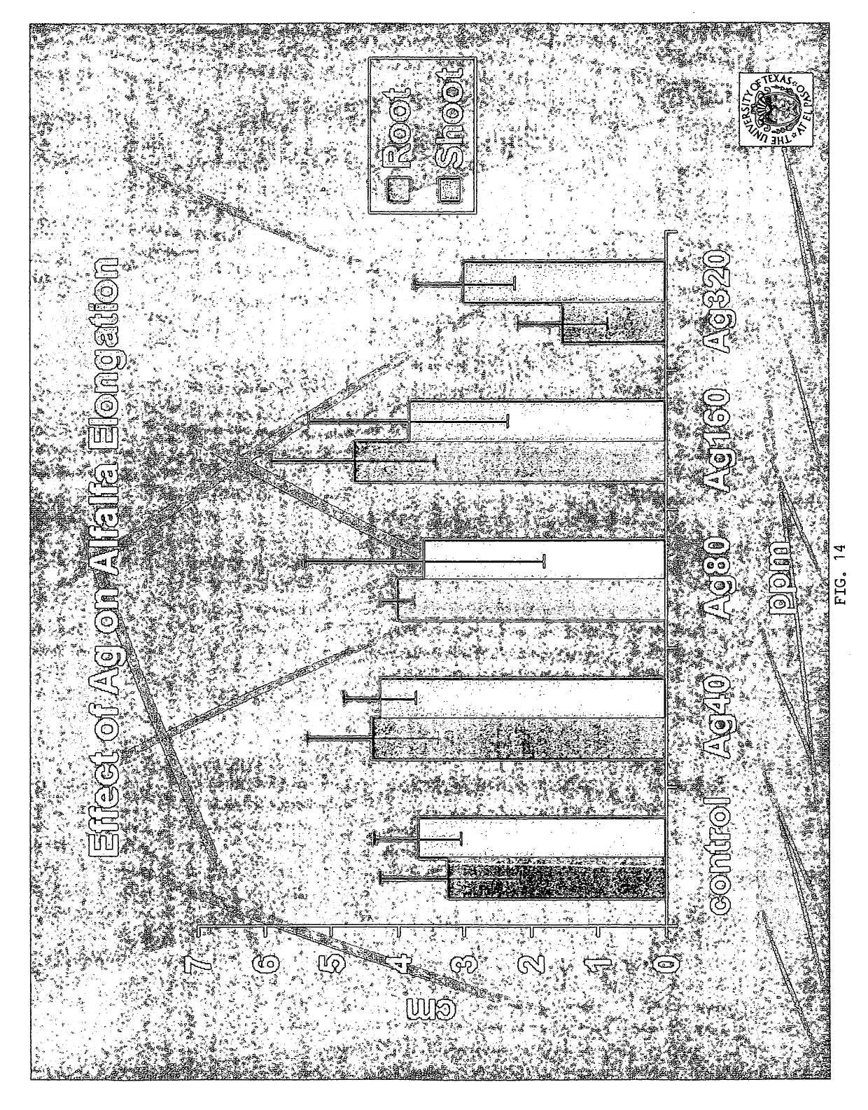

FIG. 15—Ag uptake by Alfalfa root.

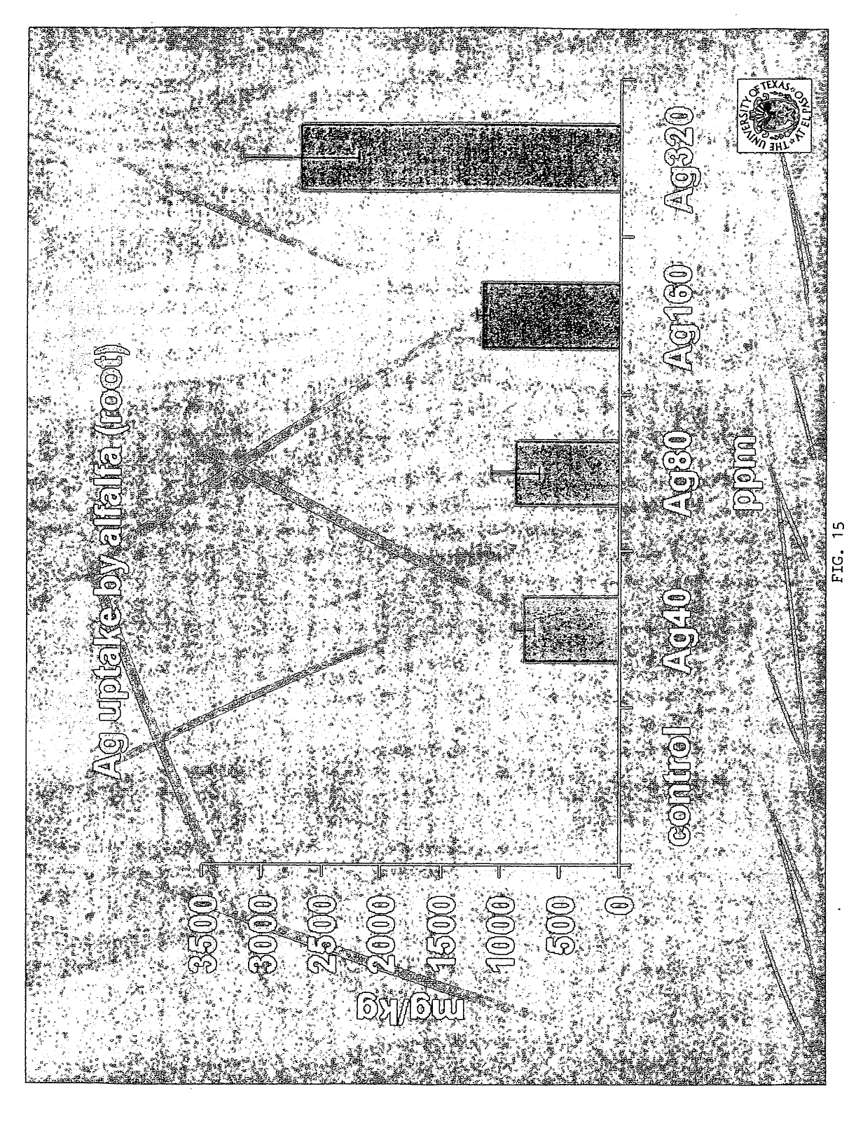

FIG. 16—Ag uptake by Alfalfa shoot.

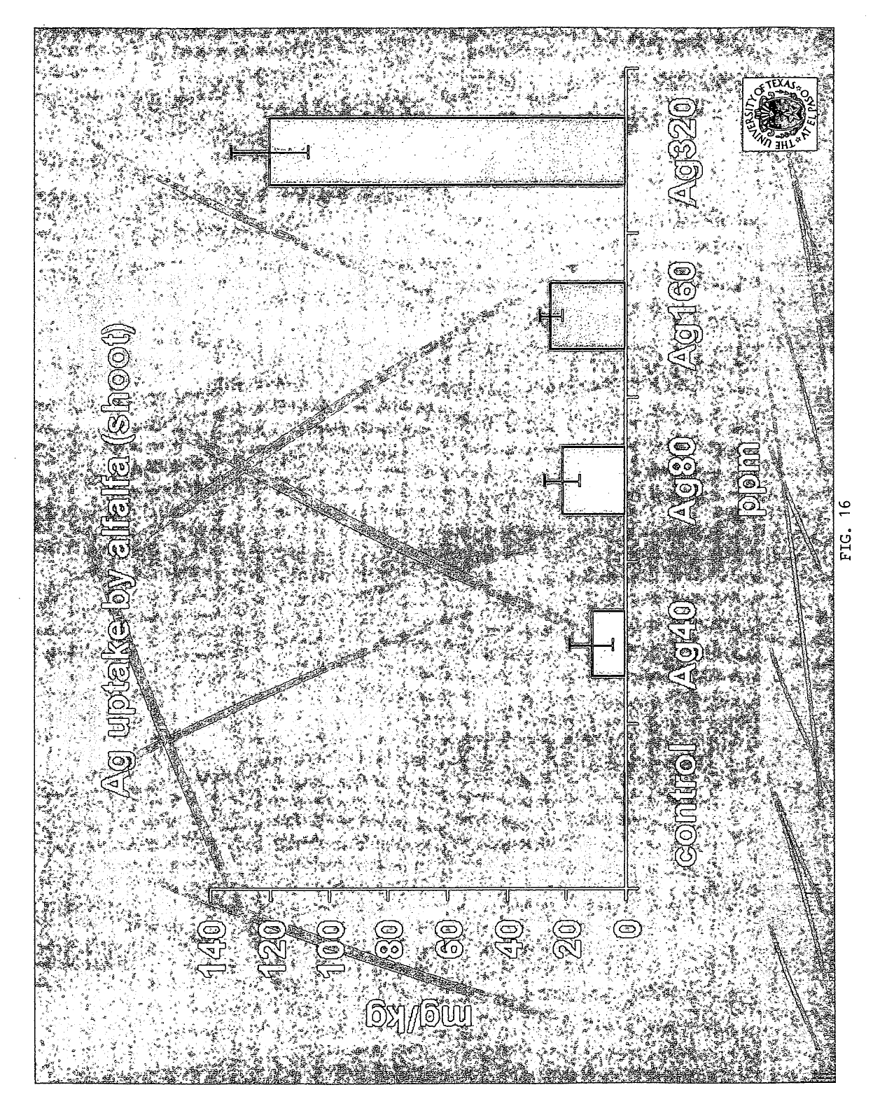

### DESCRIPTION OF ILLUSTRATIVE EMBODIMENTS

Nanoparticle technology has made a major impact on a wide variety of fields including computer science, chemistry, physics and medicine. Of course, both the source materials and methods of production for these compositions can be expensive and cumbersome. Thus, new and innovative methods are required to keep up with the demand for larger and less expensive quantities of nanoparticles, particularly those made from precious metals, such as gold, silver and platinum.

### 1. The Present Invention

The present invention involves the growth of precious metal nanoparticles inside a living plant. Using a variety of techniques to evaluate this process, such as XANES, EXAFS, X-ray EDS, TEM, and HRTEM, the inventors found that living alfalfa plants were able to uptake zero valence gold from solid media and convert it into nanoparticles. The XAS spectroscopy studies showed that the gold present in the agar and alfalfa plant samples was elemental gold. Fittings, performed using an ab initio code FEFF (v.810), showed that gold in the samples was in a structure that was only a fraction of the size needed for a complete Au(0) fcc gold particle, a result confirmed by TEM, and HRTEM. X-ray EDS and the XANES studies confirmed that the only element in the nanoparticles is gold.

The gold particles had an approximate size of 4 nm and exhibited an icosahedral structure, the lowest energy configuration for gold nanoparticles. Further analysis of the HRTEM showed the gold particles have a fcc twinned structure when the particle size ranges between 6 and 10 nm. When the particles coalesced, the fcc structure was observed. These results indicate that live alfalfa plants are a viable cost-effective means of preparing precious metal nanoparticles. This approach has important implications and applications, as discussed further below.

### 2. Nanoparticle and Nanoparticle Applications

The unique chemical, electronic, magnetic, optical, and other properties of nanoscale (sub-100 nm) particles have led to their evaluation and use in a broad range of industries, including biotechnology, catalysis, data storage, energy storage, microelectronics, and others. Today nanoparticles are used in a variety of commercial products, ranging from magnetic recording films and chemical-mechanical planarization (CMP) slurries to sunscreen products and wear-resistant coatings. The computer industry is another example of how nanoparticles can be used, such as in the preparation of microchips and chip components. Thus, the present invention contemplates a wide variety of uses for precious metal nanoparticles, although those involving electronics and optical applications are most evident.

### 3. Plants

The term “plant,” as used herein, refers to any type of plant. The inventors provide below an exemplary description of some plants that may be used with the invention. However, the list is provided for illustrative purposes only and is not limiting, as other types of plants will be known to those of skill in the art and could be used with the invention.

A common class of plants exploited in agriculture are vegetable crops, including artichokes, kohlrabi, arugula, leeks, asparagus, lettuce (e.g., head, leaf, romaine), bok choy, malanga, broccoli, melons (e.g., muskmelon, watermelon, crenshaw, honeydew, cantaloupe), brussels sprouts, cabbage, cardoni, carrots, napa, cauliflower, okra, onions, celery, parsley, chick peas, parsnips, chicory, Chinese cabbage, peppers, collards, potatoes, cucumber plants (marrows, cucumbers), pumpkins, cucurbits, radishes, dry bulb onions, rutabaga, eggplant, salsify, escarole, shallots, endive, garlic, spinach, green onions, squash, greens, beet (sugar beet and fodder beet), sweet potatoes, swiss-chard, horseradish, tomatoes, kale, turnips, and spices.

Other types of plants frequently finding commercial use include fruit and vine crops such as apples, apricots, cherries, nectarines, peaches, pears, plums, prunes, quince almonds, chestnuts, filberts, pecans, pistachios, walnuts, citrus, blueberries, boysenberries, cranberries, currants, loganberries, raspberries, strawberries, blackberries, grapes, avocados, bananas, kiwi, persimmons, pomegranate, pineapple, tropical fruits, pomes, melon, mango, papaya, and lychee.

Many of the most widely grown plants are field crop plants such as evening primrose, meadow foam, corn (field, sweet, popcorn), hops, jojoba, peanuts, rice, safflower, small grains (barley, oats, rye, wheat, etc.), sorghum, tobacco, kapok, leguminous plants (beans, lentils, peas, soybeans), oil plants (rape, mustard, poppy, olives, sunflowers, coconut, castor oil plants, cocoa beans, groundnuts), fiber plants (cotton, flax, hemp, jute), lauraceae (cinnamon, camphor), or plants such as coffee, sugarcane, tea, and natural rubber plants.

Another economically important group of plants are ornamental plants. Examples of commonly grown ornamental plants include alstroemeria (e.g., Alstoemeria brasiliensis), aster, azalea (e.g., Rhododendron sp.), begonias (e.g., Begonia sp.), bellflower, bouganvillea, cactus (e.g., Cactaceae schlumbergera truncata), camellia, carnation (e.g., Dianthus caryophyllus), chrysanthemums (e.g., Chrysanthemum sp.), clematis (e.g., Clematis sp.), cockscomb, columbine, cyclamen (e.g., Cyclamen sp.), daffodils (e.g., Narcissus sp.), false cypress, freesia (e.g., Freesia refracta), geraniums, gerberas, gladiolus (e.g., Gladiolus sp.), holly, hybiscus (e.g., Hibiscus rosasanensis), hydrangea (e.g., Macrophylla hydrangea), juniper, lilies (e.g., Lilium sp.), magnolia, miniroses, orchids (e.g., members of the family Orchidaceae), petunias (e.g., Petunia hybrida), poinsettia (e.g., Euphorbia pulcherima), primroses, rhododendron, roses (e.g., Rosa sp.), snapdragons (e.g., Antirrhinum sp.), shrubs, trees such as forest (broad-leaved trees and evergreens, such as conifers) and tulips (e.g., Tulipa sp.).

A. Dicots

In a particular embodiment, the present invention involves the use of dicotyledonous plants, or “dicots.” These plants are defined as a genus of flowering plants having two cotyledons (embryonic leaves) in the seed which usually appear at germination. Such plants may be selected from the group of tobacco, tomato, potato, sugar beet, pea, carrot, cauliflower, broccoli, soybean, canola, sunflower, alfalfa, and cotton. Of particular interest in the present invention is the use of alfalfa.

B. Alfalfa

Alfalfa (Medicago sativa L.), also called lucerne, is a herbaceous perennial legume. It originated near Iran, but related forms and species are found as wild plants scattered over central Asia and into Siberia. Its value as feed for horses and other animals was described as early as 490 B.C. by Roman writers. Alfalfa was first introduced into the eastern United States by the colonists in 1736, and thus is the oldest cultivated forage crop in the United States. It also is one palatable and nutritious, being rich in protein, vitamins, and minerals. When cut prior to bloom, it also is low in fiber and high in energy. Thus, it is prized as a primary component in dairy cattle rations and is an important feed for horses, beef cattle, sheep, and milking goats. Alfalfa is used primarily as a hay crop. It has the highest feeding value (section link) of all commonly grown hay crops when harvested at late bud or early flower stage of maturity (table link). Alfalfa also can be made into silage, pellets, meal, or cubes. With careful management, alfalfa can be used successfully as a pasture crop.

Alfalfa is worldwide in its distribution (world crop and statistical table link) and is grown in many areas of the United States, accounting for nearly 30 million acres (12 million ha) of production. A wide range of soil and climatic conditions are suitable for alfalfa, but for best production a well-drained soil with nearly neutral pH and good fertility is required. A healthy, mature alfalfa plant may have from 5 to 25 stems, which usually reach a height of 15-25 inches (38-63 cm). Stems are branched and slender and bear pinnately trifoliolate leaves. Leaves with more than three leaflets are not uncommon. Leaves are arranged alternately on the stem. Stipules are slender and adnate (fused) to the petiole. Leaflets are linear, oblong, or obovate oblong and are toothed toward their apices. Many cultivars (cultivated varieties) of alfalfa are available with specific characteristics for climatic, soil-related, insect, and disease problems. Dormant, moderately dormant, and non-dormant cultivars are available for the different climatic regions.

Alfalfa requires a deep, permeable soil with an adequate moisture supply during the growing season for maximum yields. It is very sensitive to poor drainage and compacted soil conditions that restrict root growth. Thus, alfalfa is most productive on loam or loamy soils that are both well drained and have good moisture-holding capacity. Alfalfa does not tolerate acid soils (section link) (pH below 6.2), especially in the seedling stage. A good seedbed for alfalfa is finely pulverized, leveled, and firmed to the seedling depth and contains soil moisture near the surface to initiate germination. Leveling the field to eliminate low spots will result in a more uniform stand and ease equipment travel for the life of the stand.

Most alfalfa seeding in the United States occurs either in the early spring or in late summer and fall. Time of alfalfa seeding is influenced by precipitation patterns, temperature, and cropping patterns. Spring seedings allow for harvest during the seeding year, but weed control usually is required. Late summer and fall seedings usually avoid weed competition and unfavorable summer temperatures and moisture conditions but must allow for adequate seedling development prior to the onset of winter. Spring seeding should be made early enough to allow good root systems to form before high temperature and low moisture conditions slow growth rates (seeding date program link). Late summer and fall seedings must be made early and have sufficient soil moisture to allow enough growth to minimize loss of stand from winter injury.

Alfalfa seed must be placed in contact with moist soil. Seedlings are unable to emerge from the soil if planted too deep. For best seedling survival, drill seeds approximately ¼ inch (0.6 cm) deep. Seedling emergence is greatly reduced when seeds are planted deeper than ½ inch (1.3 cm). A seeding rate of 12-15 lb/a (13-17 kg/ha) planted in 6 inch (15 cm) rows usually is sufficient for a good stand of alfalfa. Increased seeding rates normally are not economically justified when well-prepared firm seedbeds are used. However, higher seeding rates sometimes are used to compensate for poor soil preparation or seeding methods. Increase rates to 15-20 lb/a (17-22 kg/ha) for drilling and 20-25 lb/a (22-28 kg/ha) when broadcasting.

Attempts to reestablish alfalfa immediately following a previous alfalfa crop or to thicken old alfalfa stands sometimes have resulted in establishment failures. These failures have been related to autotoxicity. Autotoxicity exists when alfalfa has lower germination, poorer establishment, and/or lower production when grown immediately following alfalfa. Autotoxicity effects are attributed to plant exudates and by-products of decomposition. A period of two or more weeks between plowing and seeding or three or more weeks after herbicide killing of alfalfa and seeding can be used to avoid autotoxicity.

The presence of effective nodules on the roots of the plants is essential to a vigorous, productive stand. These nodules are formed by bacteria (Rhizobium meliloti) that are able to fix nitrogen from the air for use by the alfalfa plants. These bacteria may be present in fields where alfalfa has been grown recently, but all strains of bacteria are not equally effective. All alfalfa seed should be inoculated with a fresh commercial inoculum immediately prior to seeding, regardless of cropping history of the land or any previous inoculation of the seed. Inoculated seed should be kept cool and moist until planted.

A productive alfalfa crop is a heavy user of plant nutrients. A complete fertilizer program is essential to a long-lived stand. A soil test (fact sheet link) is the first step in planning a fertility program (section link). Applications of fertilizer and lime should be based on the results of a soil test. Lime applications are required on soils having a pH below 6.2. Apply lime well in advance of seeding, mixing thoroughly with the surface 6 inches of soil. Fall applications provide time for soil reaction to take place and avoid the spring rush that often will delay planting. Phosphorus can be applied by banding ½to 1 inch to the side or below the seed when seeding, but is most often applied by broadcasting, followed by shallow incorporation, just prior to seeding. Potassium, sulfur, and boron should be worked into the seedbed just prior to the seeding operation. Potassium and boron should not be banded near the seed. Nitrogen fertilizer is not required on legume forages because of the fixation of atmospheric nitrogen by effective nodules. Addition of nitrogen fertilizer will reduce the effectiveness of the natural nitrogen-fixing mechanism. If applied as part of other fertilizers (i.e., single ammonium phosphate), N application should not exceed 40 lb/a (45 kg/ha). Specific fertilizer recommendations based upon soil test data are provided in state Extension Service fertilizer guides (link to fertilizer guides).

Alfalfa's primary root emerges near the hilum and penetrates the soil as an unbranched taproot. As the hypocotyedonary area straightens and elongates, the cotyledonary leaves emerge aboveground. The first foliar leaf is simple (unfoliate), with a slender petiole. Subsequent foliar leaves are compound and usually trifoliolate. Vegetative buds develop in the axils of the cotylendonary leaves and subsequent foliar leaves. The timing of the removal of top growth of alfalfa by mowing or grazing has a profound influence on the productivity and length of life of an alfalfa stand. The large taproot of the alfalfa plant is a storage organ for food reserves that are needed to renew top growth in the spring and after each cutting. The maintenance of food reserves is necessary to keep the stand vigorous and productive. The amount of food reserves in alfalfa roots increases as the interval between cuttings is increased to 35 days, after which it declines. Cutting more frequently than 28 days or continuous grazing will lower the root reserve and weaken the stand.

More than 20 diseases are serious problems for alfalfa in the United States. These include fungal and bacterial wilts, leaf spots, crown and root rots, viruses, and nematodes. Important wilts are bacterial wilt (caused by Corynebacterium insidiosum), fusarium wilt (caused by Fusarium oxysporum), and verticillium wilt (caused by Verticillium alboatrum). The most serious leaf spots are common leaf spot (caused by Pseudopeziza medicaginis), lepto leaf spot (caused by Leptosphaerulina briosianna), stemphylium leaf spot (caused by Stemphylium botryosum), and summer blackstem (caused by Cercospora medicaginis). Important crown and root rots include anthracnose (caused by Colletotrichum trifolii), Aphanomyces spp. root rot, spring blackstem (caused by Phoma medicaginis), phytophthora root rot (caused by Phytophthora megasperma), rhizoctonia diseases (caused by Rhizoctonia solani), and sclerotina crown and stem rot (caused by Sclerotina trifoliorium sensu). Alfalfa mosaic (Alfalfa Mosaic Virus complex), is the primary virus disease. Alfalfa stem nematode (Ditylenchus dipsaci), root-knot nematodes (Meloidogyne spp.), and root-lesion nematodes (Pratylenchus spp.) are the most prevalent nematode species on alfalfa. Resistant cultivars are available for most of the diseases and nematodes listed.

There are a number of insects pests of alfalfa in the United States. The insect pests that interfere with forage production include the potato leafhopper, Empoasca fabae; the alfalfa weevil, Hypera postica; the spotted alfalfa aphid; the pea aphid, A. kondoi; the alfalfa plant bug, Adelphocoris lineolatus; and the meadow spittlebug, Philaenus spumarius. The potato leafhopper is the most problematic pest and causes damage throughout most alfalfa-producing areas in the eastern and central United States. It causes yellowing of the foliage and stunting of stems. The damage results in significant losses in yield and forage quality, especially loss in carotene.

### 4. Plant Environments

One aspect of the present invention involves the selection and provision of an appropriate plant environment in which to produce the nanoparticles of the present invention. This may be as simple and identifying the appropriate field in which to plant the host organism. By appropriate, one means that both the requisite precious metal (and precious metal content) is available, and that the nutrient and pH requirements are suitable for the host.

Studies by the inventors have determined that environments contains up to and including 320 ppm gold, and 320 ppm silver, though having a negative effect on shoot and root elongation (as compared to lower concentrations), are tolerated by the plant. Moreover, there is a direct correlation between environmental concentration of the metal, and the concentration (mg/kg) in the shoot and root. Thus, suitable concentrations in agar environments comprise 40-320 ppm. Field environments may have lower amounts, but could fall within the same range as well.

A. In Vitro Environments

The present invention may, in one embodiment, involve the use of in vitro or laboratory environments. Clearly, this will not permit the large scale production of nanoparticles that may find use in certain contexts. On the other hand, the ability to manipulate environmental conditions will be much greater in this sort of highly controlled environment, and thus the loss in capacity may be more than made up for by the exquisite sensitivity of the system.

In one embodiment, the host plant may be grown in a liquid environment, or “hydroponically.” This represents the height of control as the ability to alter concentrations of metals and/or nutrients in liquid is quite simple. For example, plants may be suspended from an inert scaffold, in connection with a chamber through which the liquid is circulated. The source of the fluid may have various connectors permitting the regulated introduction of components such as the metal and nutrients.

In another embodiment, the environment may be solid, such as soil or agar. Again, the use of specific mixtures allows for the very precise creation of an environment that has very particular concentrations of individual components. The nutritional components will vary, obviously, based upon the needs of the particular host plant. Determining these issues is well within the capabilities of the skilled artisan.

B. Field Environments

In another embodiment, one may select a field environment that is suitable for production. The field may, for whatever reason, contain appropriate amounts of metals. If the appropriate amounts of metals are not present, the field may be seeded using standard methodology (e.g., tillers, spraying). In this regard, of interest will be determining the concentration of metals in soil. Standard technology such as soil digestion using the EPA's 200.7 method, microwave digestion (pre-program 3051 method) and ICP-OES are contemplated.

### 5. Isolation Methods

In one aspect of the invention, one will desire to isolate the nanoparticles of the present invention. “Isolate,” this context, means increasing the content of the nanoparticles as they exist in nature. Thus, the isolation may merely involve the removal of the plant from the field, or it may involve more specific isolation comprising harvesting particle parts of the plant.

However, in more particular embodiments, the nanoparticles themselves will be purified substantially away from other plant material. The content of the nanoparticles in a given composition may be 5%, 10%, 15%, 20%, 25%, 30%, 35%, 40%, 45%, 50%, 55%, 60%, 65%, 75%, 80%, 85%, 90%, 95%, 96%, 97%, 98%, 99%, 99.5% or 100%. The degrees of purification may also be described in terms of “-fold” purification, such as 2-fold, 3-fold, 4-fold, 5-fold, 10-fold, 20-fold, 50-fold, or 100-fold.

A. Harvesting

Crop harvesting techniques are well known to those of skill in the art. Depending on the type of plant and plant part to be obtained a variety of standard techniques may be applied, including but not limited to picking and combine harvesting.

B. Physical Methods

Physical methods may be used to obtain plant-derived nanoparticles. Obviously, one such technique simply involves separating plants parts of higher particle concentration from those that do not. Of particular interest are roots, shoots and leaves. The isolation may further comprise pressing, grinding, or sonication of plant material to release nanoparticles. In addition, burning of plant material will release particles. Moreover, any of these methods may combined to improve the yield. Other physical methods address isolation at a more refined level. For example, samples may be subjected to ultrafiltration, electrophoresis (agarose, polyacrylamide) or centrifugation (density, equilibrium). Various techniques for each of these methods are available

An important physical separation method is chromatography. Any of a wide variety of chromatographic procedures may be employed according to the present invention. For example, thin layer chromatography, high performance liquid chromatography, paper chromatography, affinity chromatography or supercritical flow chromatography may be used to effect separation of nanoparticles.

Partition chromatography is based on the theory that if two phases are in contact with one another, and if one or both phases constitute a solute, the solute will distribute itself between the two phases. Usually, partition chromatography employs a column, which is filled with a sorbent and a solvent. The solution containing the solute is layered on top of the column. The solvent is then passed through the column, continuously, which permits movement of the solute through the column material. The solute can then be collected based on its movement rate. The two most common types of partition chromatograph are paper chromatograph and thin-layer chromatograph (TLC); together these are called adsorption chromatography. In both cases, the matrix contains a bound liquid. Other examples of partition chromatography are gas-liquid and gel chromatography.

Paper chromatography is a variant of partition chromatography that is performed on cellulose columns in the form of a paper sheet. Cellulose contains a large amount of bound water even when extensively dried. Partitioning occurs between the bound water and the developing solvent. Frequently, the solvent used is water. Usually, very small volumes of the solution mixture to be separated is placed at top of the paper and allowed to dry. Capillarity draws the solvent through the paper, dissolves the sample, and moves the components in the direction of flow. Paper chromatograms may be developed for either ascending or descending solvent flow. Two dimensional separations are permitted by changing the axis of migration 90° after the first run.

Thin layer chromatography (TLC) is very commonly used to separate lipids and, therefore, is considered a preferred embodiment of the present invention. TLC has the advantages of paper chromatography, but allows the use of any substance that can be finely divided and formed into a uniform layer. In TLC, the stationary phase is a layer of sorbent spread uniformly over the surface of a glass or plastic plate. The plates are usually made by forming a slurry of sorbent that is poured onto the surface of the gel after creating a well by placing tape at a selected height along the perimeter of the plate. After the sorbent dries, the tape is removed and the plate is treated just as paper in paper chromatography. The sample is applied and the plate is contacted with a solvent. Once the solvent has almost reached the end of the plate, the plate is removed and dried. Spots can then be identified by fluorescence, immunologic identification, counting of radioactivity, or by spraying varying reagents onto the surface to produce a color change.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

The gel material for gel chromatography is a three-dimensional network whose structure is usually random. The gels consist of cross-linked polymers that are generally inert, do not bind or react with the material being analyzed, and are uncharged. The space filled within the gel is filled with liquid and this liquid occupies most of the gel volume. Common gels are dextran, agarose and polyacrylamide; they are used for aqueous solution.

High Performance Liquid Chromatography (HPLC) is characterized by a very rapid separation with extraordinary resolution of peaks. This is achieved by the use of very fine particles and high pressure to maintain and adequate flow rate. Separation can be accomplished in a matter of minutes, or a most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

C. Chemical Methods

Chemical methods involve the use of chemical species to either disrupt plant material, or to segregate various materials to enhance the purity of the nanoparticles. One example is base or acid hydrolysis of plant material, which can disrupt cells and cell walls. It is envisioned that such a technique could be combined, advantageously, with physical methods such as grinding, pressing, or burning, and may further be improved by mixing of the plant material and chemical. Alternatively, chemical separation may involve the use of different chemicals to “partition” particles away from plant material. Such partitioning may rely on phase separation between organic an inorganic materials. Due to their chemical similarity or dissimilarity with plant materials and particles, suitable chemicals may be useful in separating of metal nanoparticles, such as dimethyl fluoride, tetrabutyl ammonium carbonyl, toluene, and benzene.

D. Biologic Methods

Biologic methods, in the context of the present invention, primarily involve the use of microbes or enzymes that degrade plant materials. There are a number of microbial agents that produce enzymes capable of digesting plant materials. These microbes, or enzymes derived therefrom, may be utilized to disrupt plant material so as to release and isolate metal particles. Examples include collagenases, chitinases, cutinases, lipases, pectases, (endo)glucanases, proteases, DNAses, RNAses and cellulases. And while a primary source of enzymes will be from microbes, mammalian enzymes may be used as well. Again, biologic methods may be combined with chemical and/or physical disruption.

### 6. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Gold Methods

XAS studies were performed on alfalfa plants grown on a gold rich agar system. XANES were used to observe the oxidation state of the gold in both the plants and the agar. EXAFS were used to investigate the nearest neighboring atom to the gold in the plants and the agar. Finally, studies were performed using X-ray EDS, TEM, and HRTEM to further investigate the formation of gold nanoparticles in the plants.

X-ray absorption spectroscopy (XAS) was used in this study to gain information about the uptake and formation of gold nanoparticles by live alfalfa plants. Koningsberger and Prins (1988); Gardea-Torresdey et al. (2000). X-ray absorption near edge structure (XANES) and extended X-ray absorption fine structure (EXAFS) are two components of XAS, which provide information about the oxidation state, coordination environment, and the nearest neighboring atom.

The alfalfa seeds of the Mesa variety were used and prepared similar to that of Peralta et al. (2001). The seeds were soaked to avoid fungal contamination in 3% formaldehyde for 15 min and washed three times with deionized water. Approximately 100 seeds were transferred to a mason jar of one-pint volume already autoclaved for a sterile environment. Each jar contained 200 ml of nutrient solution made with Ca(NO₃)₂,4H₂O (3.57×10⁻⁴M), H₃BO₃ (2.31×10⁻⁵M), CaCl₂,2H₂O (2.14×10⁻³M), KH₂PO₄ (9.68×10⁻⁴M) KNO₃ (2.55×10⁻⁴M), MgClO₄ (1.04×10⁻³M), FeCl₃ (6.83×10⁻⁵M), MnSO₄,H₂O (7.69×10⁻⁶M), MoO₃ (1×10⁻⁵M), ZnSO₄,7H₂O (4.3×10⁻⁵M), CuSO₄,5H₂O (1.6×10⁻⁶M), and agar-agar, 1 g per 200 mL. Gold(III) from potassium tetrachloroaurate was used at concentrations of 0, 5, 10, 20, 40, 80, 160, and 320 ppm. All jars were covered with plastic wrap after alfalfa seeds were spread out to avoid contamination from the environment and to allow light to pass through. Four replicates of each treatment were prepared at the optimum pH of 5.8, for quality control and quality assurance purposes. The study was performed in a growth chamber under a 12 h photoperiod, and 25/18° C. day/night temperature variations. The alfalfa plants were harvested after 2 weeks of growth, and the alfalfa plants were washed three times with deionized water. For the XAS experiments, the washed alfalfa samples and the agar samples were frozen in liquid nitrogen for 40 min and then placed in a Freeze-Dry System for 2 days to remove any water within the samples.

XAS were conducted at the Stanford Synchrotron Radiation Laboratories (SSRL). The Au L(III) edge (11.918 keV) was used for collecting the sample spectra using standard operating conditions of 3 GeV and 60-100 mA, Salt et al. (1999). The gold(III)-loaded alfalfa plant samples were run at approximately 20 K using a liquid helium cryostat to reduce interference in the spectra of the sample due to thermal disorder. The fluorescence spectra of the alfalfa samples were acquired using a 13-element array germanium detector. The transmission measurements for the gold model compound were taken using argon-filled ionization chambers. The model compound tetrachloroaurate was ground and diluted using boron nitride to achieve a composition to give a change of 1 absorbance unit across the absorption edge. The samples and model compound were packed into 1 mm aluminum sample holders with X-ray transparent tape. A Si220 double crystal monochromator with an entrance slit of 1 mm was used for the measurements. The monochromator was detuned by 50% to reject higher order harmonics. All spectra were calibrated against the edge position of a gold foil (11.918 keV). The WinXAS software package was used to analyze the experimental EXAFS. Peralta et al. (2001); Salt et al. (1999); Ressler (1998; 1997); Ankudinov et al. (1998). The EXAFS were extracted from the absorption spectra by subtracting a two-range spline fit to the preedge region from the total spectrum and normalizing the difference using a polynomial fit to the postedge region.

### Example 2

### Gold Results

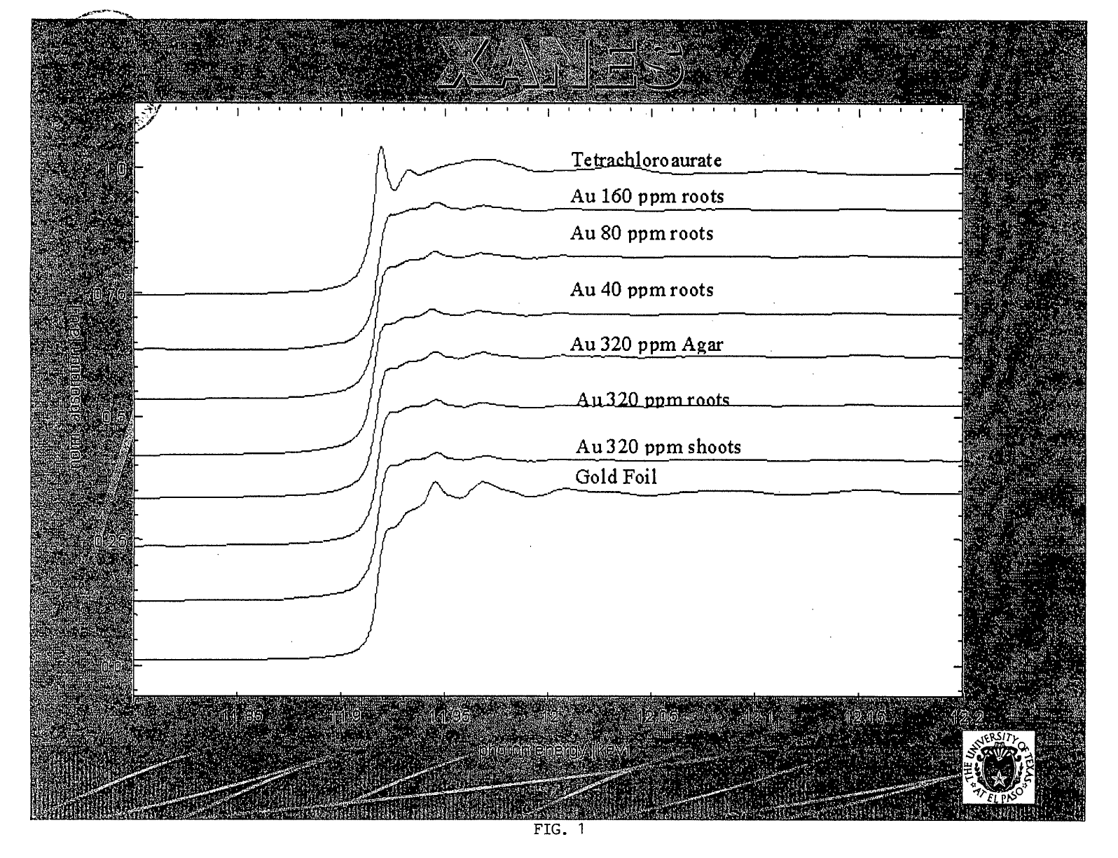

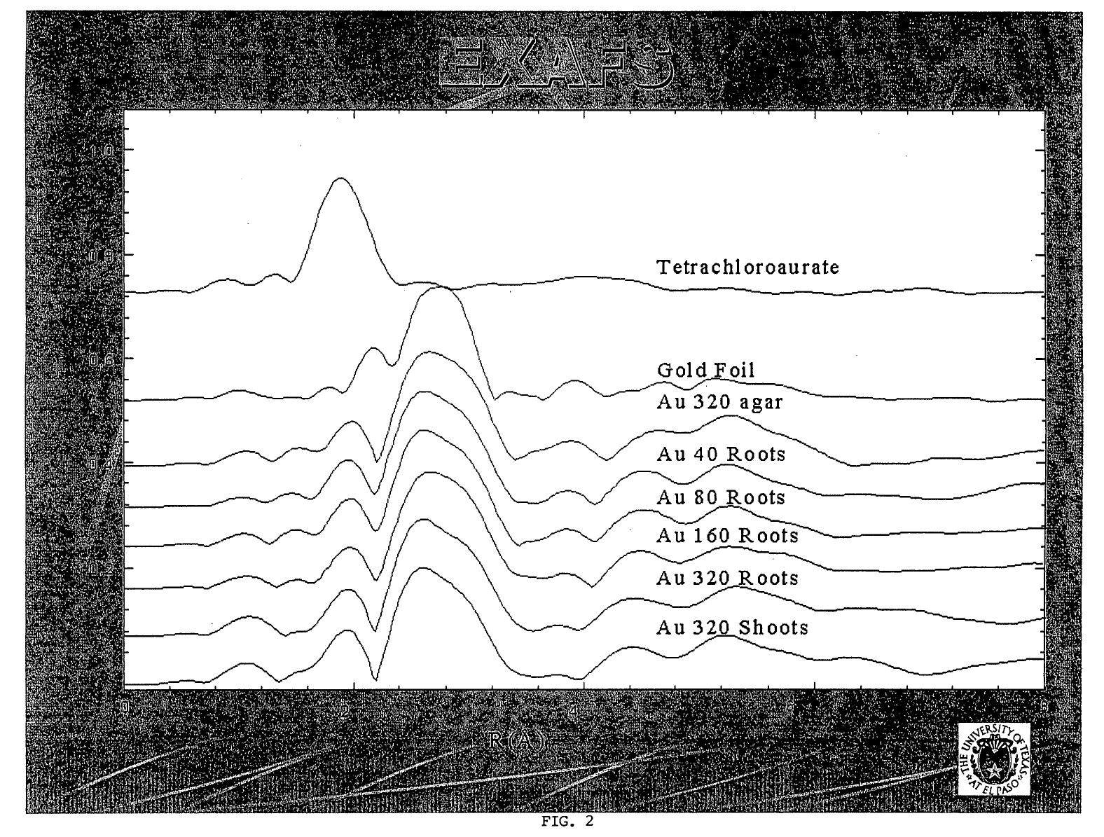

FIG. 1 shows the XANES spectra of the gold alfalfa root and shoot samples and the model compound. The edge energy or position of the plant and agar samples matches that of the gold(0) (11.981 keV) foil and not that of the initial reactant tetrachloroaurate. The position of the edge energy clearly shows that the gold present in the plant and agar is present as gold(0). Additionally, this shows that the alfalfa has the ability to actively uptake gold(0) from solid media. Furthermore, our results show that the gold(III) is being reduced to gold(0) in the agar solid media. FIG. 2 shows the EXAFS of the gold samples and the tetrachloroaurate (not phase and amplitude corrected). Ankudinov et al. (1998); Rehr and Albers (1990).

As observed the nearest neighbor in the tetrachloroaurate sample has a much shorter interatomic distance that the nearest neighbor in the gold alfalfa plant roots and shoots and gold agar samples. But, the nearest neighbor in the gold(0) foil appears at the same distance as the nearest neighbor in the gold plant and agar sample, thus confirming that the gold present in plant shoot, roots, and agar samples is gold(0). This result confirms that alfalfa has the ability to actively uptake gold(0) from a solid media.

The Au—Au bond of the gold foil has a distance of 2.86 Å and a coordination number of 12. The model compound tetrachloroaurate has, as expected, a Au—Cl bond with a distance between them of 2.28 Å and a coordination number of 4. The remaining samples all have similar bond distances as the Au—Au (within (±0.01 Å) but differ in their coordination numbers. The coordination number gives an indication that gold present in the biomass and agar samples are fractions of the total size of a full fcc Au particle.

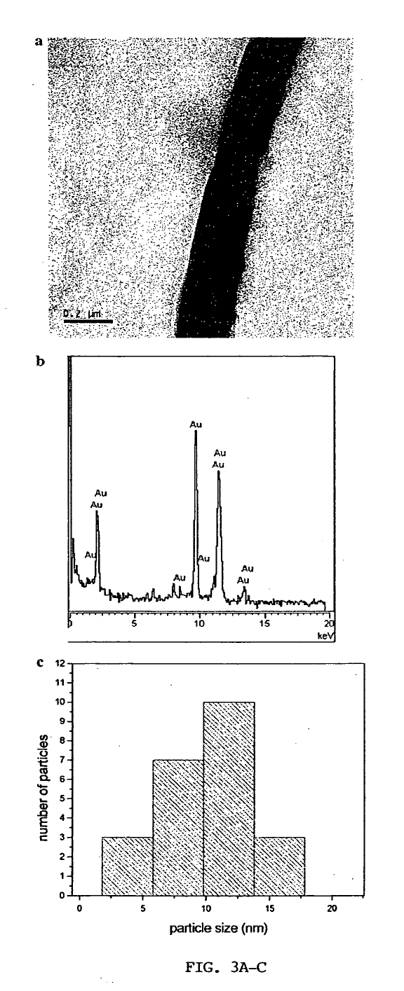

For the TEM analysis new plant samples were embedded in a synthetic resin and dried in a furnace at 60° C. for 24 h. Thin slices (˜50 nm thick) were cut using a microtome and put on Cu grids and placed on the sample holder of the TEM. The samples were not stained for the TEM analysis. As a consequence, the only possible sources of contrast are mass thickness differences or diffraction contrast between different zones and the gold nanoparticles themselves. A JEOL 2010-F TEM equipped with a X-ray EDS elemental microanalysis system was used to study the nanoparticles within the plants. FIG. 3A shows a low magnification TEM image of the alfalfa shoots, confirming the existence of Au nanoparticles aggregated together. In this image the small black dots correspond to the gold nanoparticles. This image also indicates that the nucleation of the particles inside the plants occurs in preferential zones. The morphology in FIG. 3A suggests an active transport of gold(0) atoms through the plants from the roots to the shoots. This active transport of the gold atoms is indicated by the positions of the particles throughout the entire region of the shoot in the micrograph. This confirms the EXAFS and XANES results since they showed that gold(0) was found in both the roots and shoots, although at lower concentrations in the stems, the signal was lost (data not shown). FIG. 3B is a qualitative X-ray EDS spectrum corresponding to an arbitrary nanoparticle. The analysis confirms that the nanoparticle is pure gold. The same study was performed on many different nanoparticles, confirming that the only constituent element of each nanoparticle is Au. FIG. 3C shows the particle size distribution found for all the samples. This figure shows a broad distribution with typical sizes between 2 and 20 m in diameter. The difference in size is possibly due to the fact that the nanoparticles are being formed at different times.

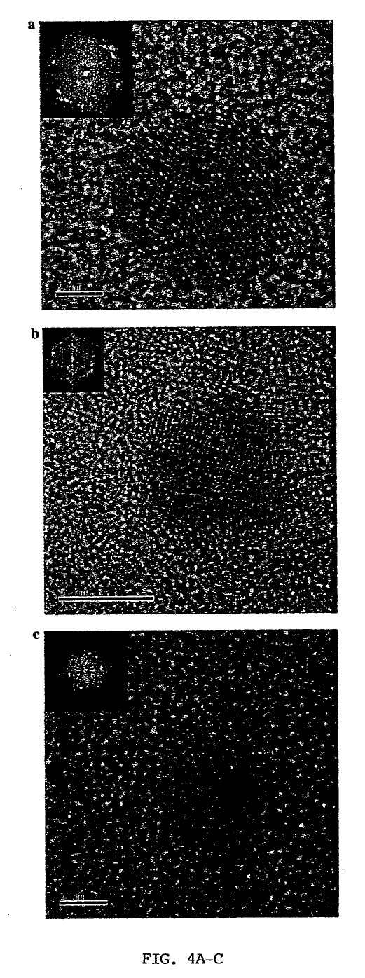

HRTEM images of the gold nanoparticles are shown in FIGS. 4A-C for three different examples. The images were obtained at the optimum defocus condition. Different particles were imaged using the high-resolution mode, showing that the nanoparticles are in a crystalline state. The inset in the three images shows the corresponding fast Fourier transform (FFT). In the case of nanoparticles <5 nm thick, the FFT is similar to the diffraction pattern and therefore it can be used to study the interplanar distances. Tomita et al. (1985). The shortest lattice parameter calculated from each image is in every case 0.23 nm. This value corresponds approximately to the interplanar spacing between (111) planes of gold. The particles have different atomic orderings and many show the presence of microstructural defects that are similar to gold nanoparticles obtained by synthetic techniques such as evaporation or colloidal methods. Yacaman et al. (2001). The splitting of the points observed in the FFT confirms that the particles are intertwinned. In FIG. 4C, a very small crystalline nanoparticle can be observed in the first stage of the nucleation process.

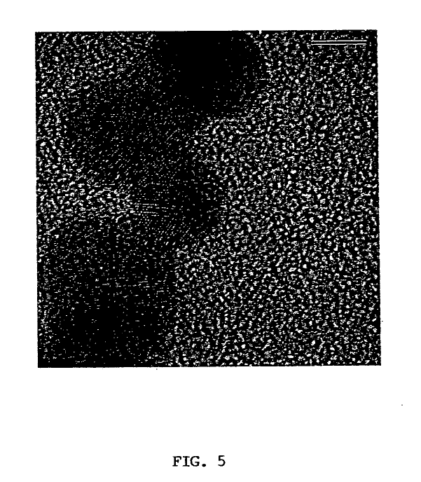

A closer examination of the FFT pattern indicates that there are spots showing splitting of the particles. By measuring the angle of splitting and comparing with previous calculations, we concluded that the structure corresponds to a icosahedron tilted away about 18° out of the 3-fold orientation. Urban et al. (1993). Such a small particle with an icosahedron structure implies that the gold atoms arrange themselves into the minimum energy configuration even within the plant. FIG. 5 shows the coalescence of the nanoparticles within the plant structure. The image shows atomic resolution in all the particles, suggesting an orientational relationship among the different particles. Additionally, within the same plant sample, it was possible to find small particles of approximately 4 nm and large coalesced particles ranging in size between 20 and 40 nm. This result clearly indicates that continuous growing and particle rearrangement processes occur within the plant structure.

The nucleation of Au nanoparticles inside living plants is a remarkable phenomenon, which deserves a deeper study in relation with possible applications. It offers three interesting possibilities: (i) it provides a new method for the synthesis of gold nanoparticles; (ii) it will generate new studies concerning the interaction between the plants, metals, and metal ions; (iii) it may constitute a new method for the mining of gold from solution and soils as an inexpensive method for the purification of economically important elements.

### Example 3

### Silver Methods

Mesa variety alfalfa seeds were immersed for 30 minutes in a Captan solution (2 g/L) in order to avoid fungal contamination and then washed three times with sterilized (autoclaved) deionized water. Under a control air hood (Laminar flow hood) and a sterile environment, seeds were transferred to mason jars containing 75 ml of agar solidified nutrient media. The nutrient media contained both the macro and micronutrients that the plants require to grow. For this study, the inventors modified the media previously described in the literature (Peralta et al., 2001). The only chloride salts added to the medium were MnCl₂.4H₂O and FeCl₃; the other chloride salts were respectively substituted by either their nitrate or sulfate salts to avoid silver chloride precipitation. A 5 g mass of Difcobacto™ agar was added to every liter of nutrient solution prepared.

The source of silver ions in this study was supplied as silver nitrate (AgNO₃) and the concentrations used were 0, 40, 80, 160, and 320 mg of silver per liter of solution (or parts per million, ppm). Before the addition of agar, the media was adjusted to pH 5.8 (the physiological pH of alfalfa). After the alfalfa seeds were spread out, all jars were covered with plastic wrap previously treated with formaldehyde (3% v/v) in order to avoid contamination and permit light to pass through it. After planting, all jars were set in a growth chamber for a 12-hour light/12-hour dark photoperiod at 25° and 18° C., respectively. Four replicates of each treatment were prepared for quality control and statistical purposes.

The alfalfa plants were harvested 9 days after germination. After harvesting, a sample of each treatment was immersed in 0.01M HCl for 10 seconds, washed three times with deinoized water, and then immersed along with an agar sample in liquid nitrogen for 40 minutes. Finally, samples were placed in a freeze-dryer (Labconco, Freeze drying system/Freezone 4.5) for two days. The purpose of this step was to dehydrate and eventually pulverize the biomass into a fine homogeneous powder using a pestle and mortar (Lytle et al., 1998).

These alfalfa and agar samples were taken to Stanford Synchrotron Radiation Laboratories (SSRL) for X-ray absorption spectroscopy, including X-ray absorption near edge spectroscopy (XANES) and extended X-ray absorption fine structure (EXAFS) of the Ag K edge (25.514 keV). This analysis was performed in order to investigate the oxidation state, the interatomic distance, and number of nearest neighbor atoms to the silver atoms in the different parts of the alfalfa plants and the agar (Penner-Haln, 1999). The typical operating condition was a beam current of 60-100 mA and energy of 3 GeV. All samples were run at a temperature of about 15° K, using a helium cryostat, to reduce Debye Waller effects which arise from thermal disorder in samples. Fluorescence spectra of samples were taken with the aid of a 30-element Canberra germanium detector. Transmission measurements were recorded for the model compound silver nitrate using argon filled ionization chambers. The model compound was diluted in boron nitride to give a change of one absorption unit across the edge. The model compound was diluted and homogenized with the boron nitride using a pestle and mortar prior to analysis. A silicon (220, at φ90) double crystal monochromator with an entrance slit of 1 mm was used for all measurements. The monochromator was detuned by 50% for the rejection of higher harmonics. Several scans were averaged for XANES and EXAFS spectra in order to improve the signal to noise ratio. All samples were packed in 1 mm aluminum holders with X-ray transparent tape and measured as solids (Gamez et al., 2002). All spectra were calibrated against the edge position of an internal standard silver foil (25.514 keV).

The WinXAS software was used to analyze the data through standard methods (Ressler, 1998). The samples were calibrated against the silver edge energy using 1^{st} and 2^{nd} degree derivates of the foil edge energy (25.514 keV). The sample spectra were then background corrected using a 1 degree polynomial on the pre-edge region and a 5^{th} degree polynomial on the post-edge region. The XANES region was then extracted from the entire spectra, from 25.45 keV to 25.70 keV. This was then fitted using a LC-XANES (linear combination XANES) fitting of the model compound silver nitrate. The conversion into K space is based on the kinetic energy photoelectrons ejected at the edge energy of keV, using 1^{st} and 2^{nd} derivatives of the edge energy of the sample. The EXAFS spectra were extracted by taking a spline of 7 knots of the spectra and converting the spectra into k space (or wavevector space). The resulting EXAFS spectra were then Fourier transformed in a modified Hanning window over the first and last 10% of the entire EXAFS spectra between 2 and 12.2 Å and subsequently k weighted to 3. The fitting of the EXAFS was performed using least squared fitting of the Fourier filtered spectra, using an ab initio multiple-scattering code FEFF V 8.00 (Ankudinov et al., 1998). The FEFF input files were created using the ATOMS software from crystallographic data of the model compounds (Ravel, 2001). From the ab initio calculations of FEFF, the number of coordinating atoms, the nearest neighboring atoms, and the Debye Waller factors were calculated.

The alfalfa plant samples grown in a silver rich media (40, 80, 160, and 320 ppm) were embedded in a synthetic resin and dried in an oven at 65° C. for 24 hrs. Thin slices of plant shoots ranging from 40 to 90 nm in width, were cut in a microtome and put on Cu grids covered with a thin (few nm) carbon layer commonly used for TEM observations. TEM analysis was performed using a JEOL 2010-F TEM equipped with field emission gun and an X-ray EDS elemental microanalysis system, which allowed analysis of the composition of isolated nanoparticles and a STEM unit with a high angle annular dark field detector (HAADF). The samples studied in the TEM were non-stained samples. In consequence, the only possible sources of contrast for these conditions were mass-thickness, diffraction contrast, and phase contrast for TEM normal operation.

### Example 4

### Silver Results

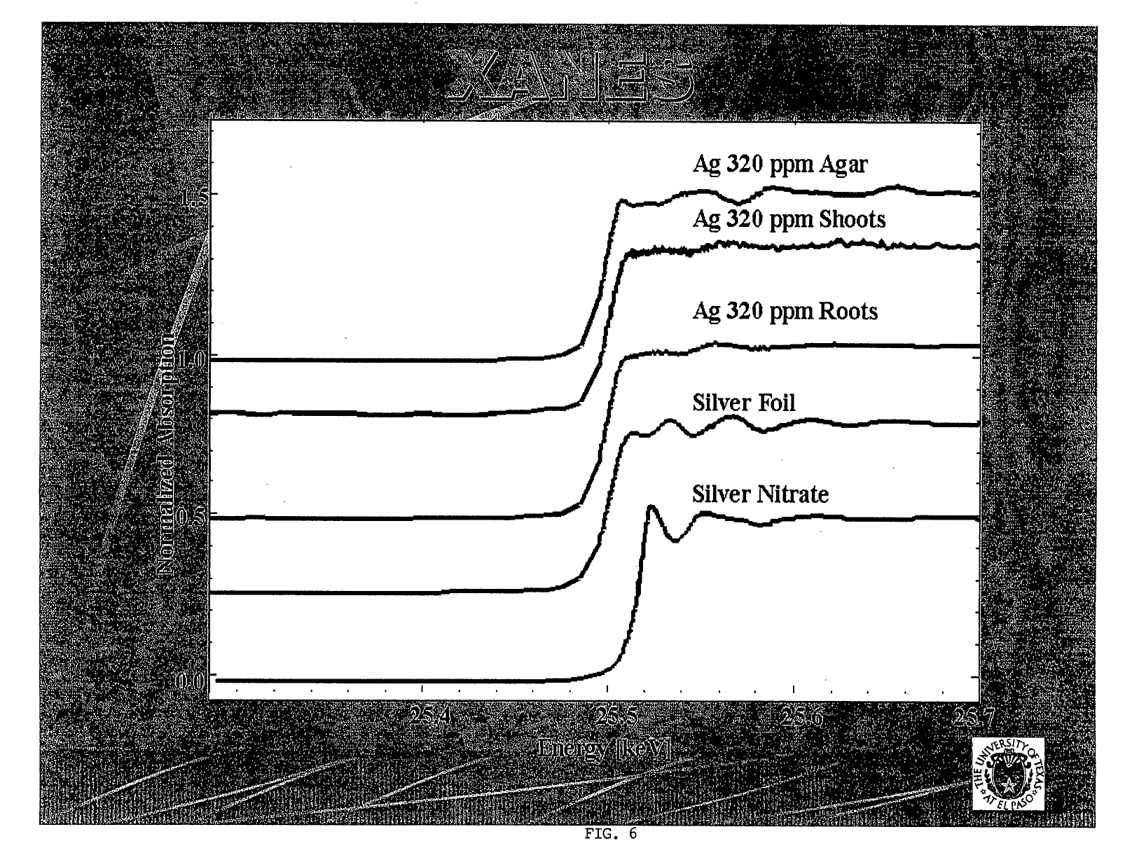

FIG. 6 shows the XANES spectra of the silver alfalfa root and shoot samples, the agar, the model compound (AgNO₃), and the internal standard silver foil. The silver nitrate absorption edge appears at a higher energy than the silver foil due to the oxidation state of Ag(⁺¹). Since the atom had lost a “d” electron in silver nitrate, the “1s” electrons are de-shielded from the nucleus, therefore increasing their binding energy. The spectra obtained from the roots, shoots, and agar are very similar and their absorption edges appear at the same energy of that of the silver foil. From this information, it is apparent that the Ag⁺¹ ions are being reduced in the solid media, absorbed by the roots and transferred to the shoots as Ag(0). The same phenomenon has been observed for gold since Au(III) ions were found to be reduced in agar, absorbed by the roots as Au(0), and transferred to the shoots in that oxidation state (Gardea-Torresdey et al., 2002). The linear combination fitting analysis (LC-XANES analysis) (Table 1) showed that more than 95% of the silver found in the roots, shoots, and agar was present as Ag(0). The remaining percentage of silver was present as Ag(I).

TABLE 1
Results from Linear Combination Fitting Analysis*

| Sample | % Ag(I) Nitrate | % Ag(O) Foil |
|---|---|---|
| Ag Agar 320 | 1.5 | 98.5 |
| Ag Roots 320 | 5.1 | 94.8 |
| Ag Shoot 320 | 3.3 | 96.7 |

*This table shows percentages of Ag(O) and Ag(I) present in the different samples.

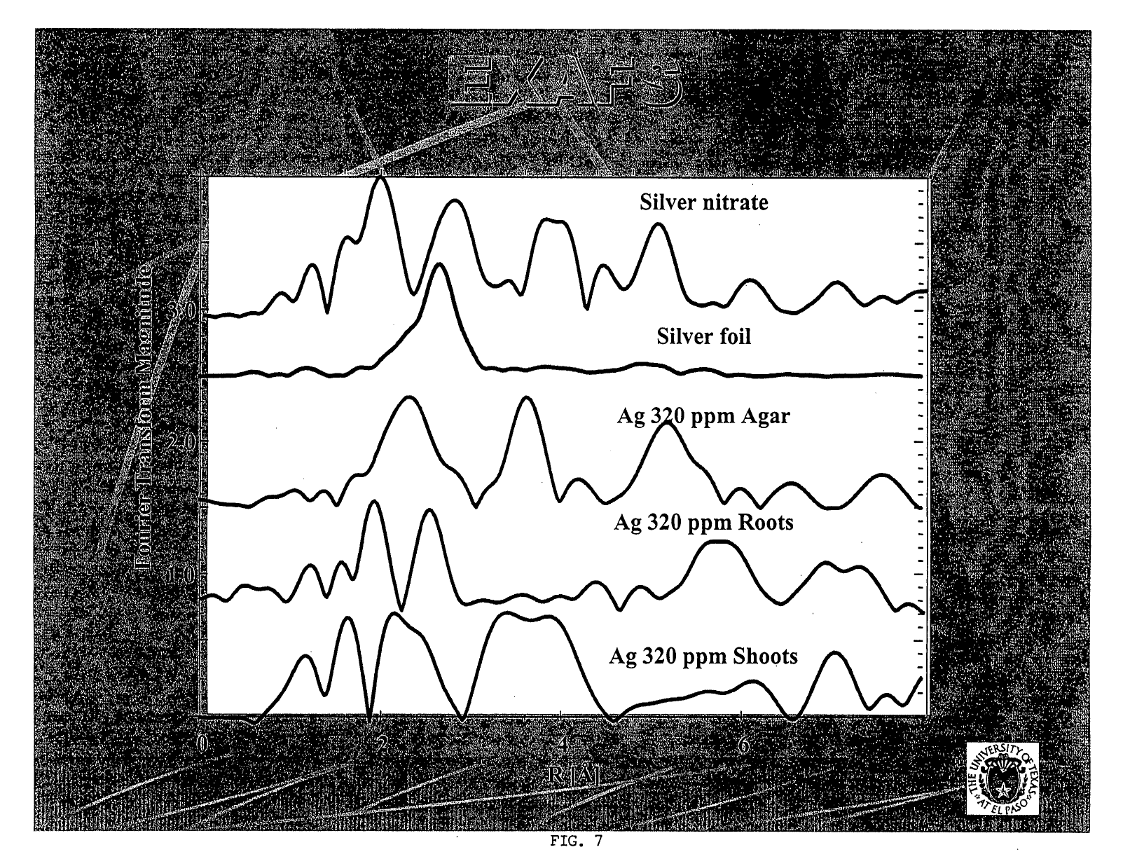

FIG. 7 shows the EXAFS of the silver samples, the agar, the model compound (AgNO₃), and the internal standard silver foil. From these measurements, the information obtained is the interatomic distances from nearest neighbors and the number of nearest neighboring atoms to the silver atoms (also shown in Table 2).

TABLE 2
EXAFS results showing the type of bond, coordination number (N),
interatomic distances (R(Å)) and mean square relative displacement
(σ²(Å)) from the silver atom to the nearest neighbors of the different
samples analyzed

| Sample | Bond | N | R(Å) | σ²(Å) (±) |
|---|---|---|---|---|
| Silver(0) | Ag—Ag | 10.8 | 2.87 | 0.0092 |
| Silver(I) Nitrate | Ag—O | 2.4 | 2.48 | 0038 |
| Silver Alfalfa Roots 320 ppm | Ag—Ag | 4.3 | 2.88 | 0.016 |
| Silver Alfalfa Shoots 320 ppm | Ag—O | 1.0 | 2.12 | 0.0067 |
| | Ag—Ag | 1.0 | 2.57 | 0.0040 |
| Silver Agar 320 ppm | Ag—Ag | 8.9 | 2.71 | 0.0137 |
| | Ag—Ag | 7.5 | 2.41 | 0.0060 |

The nearest neighbor of the silver nitrate sample has an interatomic distance of 2.48 Å, which is shorter when compared to the silver alfalfa roots and agar samples due to the Ag—O bond. Whereas the interatomic distance obtained from the Ag(0) internal standard foil appears at a distance of 2.87 Å, very similar to the nearest neighbors in the silver alfalfa root. However, the silver alfalfa root sample also shows an Ag—Ag multiple scatter peak at approximately 1.98 Å (FIG. 7). Montano et al. (1989) have shown that this is indicative of a 9 Å silver particle. The silver agar sample shows a small contraction in the nearest neighbor distances of about 0.16 and 0.46 Å for the Ag—Ag bond. Meanwhile, in the silver alfalfa shoot, there is a noticeable decrease in bond length of 0.3 Å for the Ag—Ag bond and 0.36 Å for the Ag—O bond, suggesting the presence of silver dimers with a diameter of about 5.14 Å. This feature of nearest neighbor interatomic distance contraction has been previously reported (Montano et al., 1989). These contractions are due to the silver particle size. Silver clusters averaging more than 20 Å in diameter show the contraction phenomenon that can be explained in terms of surface stress. However, silver clusters with a mean diameter of 16 Å undergo a small expansion. Furthermore, it was possible to detect interactions up to the 5^{th} shell (FIG. 7) equivalent to more than 6 Å of interatomic distance (Montano et al., 1989). Therefore, these results suggest that silver in the roots are present with a particle size of 9 Å and are eventually transported as silver nanoparticles. In addition, atomic dimers or multimers are formed in the shoots. This could be an important finding since this appears to be the first time that silver dimers, multimers, or atomic wires are reported to be produced within a living plant.

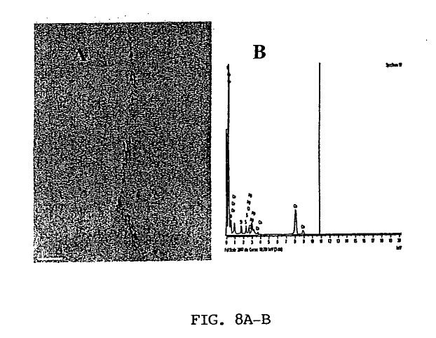

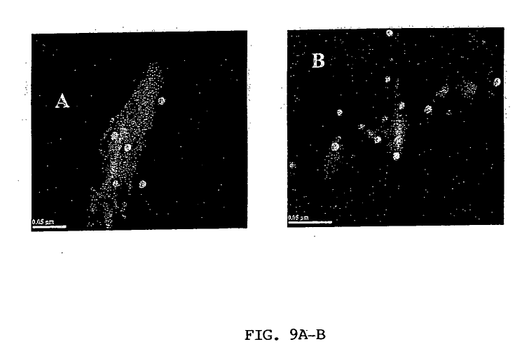

FIG. 8A displays a low magnification TEM image showing a set of Ag nanoparticles arranged approximately in a one-dimensional structure. The small black dots in the image correspond to the Ag nanoparticles. The image also indicates the zones in the plant that Ag ions go through. X-ray EDS analysis (FIG. 8B) was performed on individual particles, indicating in all cases that the constituent element was Ag. Other elemental signals were recorded possibly due to emissions from proteins or enzymes present in the biomass (Priyabrata et al., 2001). HRTEM experiments were performed in the nanoparticles to discern the detail of the atomic and defect structure. In FIG. 9A, one can observe a small nanoparticle whose size is between 2 to 3 nanometers in diameter. This image corresponds to one of the smallest nanoparticles found showing a crystalline state in an icosahedral structure (Gardea-Torresdey et al., 2002). The morphology of the silver nanoparticles obtained in this study is mostly spherical, differing from the ones obtained by Klaus et al., who obtained nanoparticle morphologies with spherical, triangular, and hexagonal appearance from silver-resistant bacteria (Klaus et al., 1999). Furthermore, the size of the silver nanoparticles ranges from 2 to 20 nm in diameter, differing from the size of particles obtained by Priyabrata et al. using the fungal Verticillium cells, who observed nanoparticles of 12 to 35 nm in diameter (Priyabrata et al., 2001).

The TEM images show dark contrast among different nanoparticles. Therefore, the same samples were investigated using STEM. The best images were obtained using the Z-contrast high angle annular dark field detector (HAADF), in which the contrast among different zones is based mainly on atomic mass (Z) differences. The intensity in a certain region is proportional to Z (Rao and Cheetham, 2001). In the STEM image, it is very easy to discern the very bright zones from the completely dark zones (FIGS. 9A and 9B). In the bright zones, it is possible to distinguish the nanoparticles as well as zones in-between different nanoparticles, constituted by Ag in a non-crystalline state but connecting the nanoparticles to one another. The smaller particles may represent the silver atomic wires or clusters of dimers, as mentioned previously, and the dark field image TEM data corroborate our EXAFS results.

From the TEM analysis, different and interesting observations were made regarding particle alignment, structure, and nanoparticle coalescence. As far as particle alignment, the particles are usually found in specific areas in the plants. These areas are possibly related to the internal anatomy of alfalfa stems. The plants absorb Ag atoms through specific channels and, consequently, the Ag nucleates or coalesces as particles inside these channels. However, we also found nanoparticles outside the channels. This observation was either due to a collapse in the plant structure, allowing the silver nanoparticles to move out of these original areas and possibly, due to silver diffusing, and nucleating nanoparticles in different places.

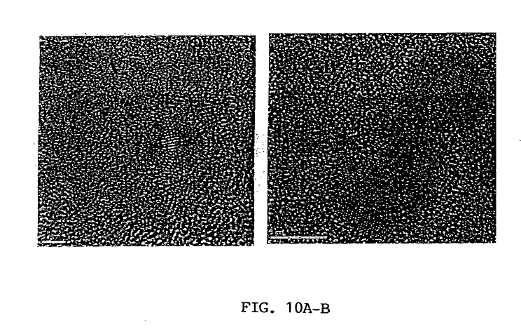

As for the structures, nanoparticles are present in a crystalline state as indicated by HRTEM images (FIGS. 10A and 10B). The nanoparticles offer an interesting variety of crystal structures. Silver nanoparticles ranging between 2 to 4 nm in size were found to have an icosahedral structure (FIG. 10A). This structure corresponds to the lowest energy configuration for this size (Gardea-Torresdey et al., 2002). In addition, the Ag nanoparticles showed, in many cases, internal defects (twinning, mixed structures, dislocations). The arrangement of atoms inside the nanoparticles was very disordered. This indicates that the aggregation process for this phenomenon could be very fast and or not under equilibrium conditions.

FIG. 10B also shows the coalescence of different nanoparticles. This phenomenon was observed in various cases for the alfalfa samples. Different nanoparticles share a common lattice when the nanoparticles coalesced. This means that the individual nanoparticles rearrange their crystal structure when they coalesce. The elimination of internal boundaries, defects, and the same crystalline orientation of the final product is a minimal free energy situation.

All of the composition and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### 7. REFERENCES

The following references, to the extent that they provide exemplary procedural or other details supplementary to those set forth herein, are specifically incorporated herein by reference.
Ankudinov et al., Phys. Rev., B:7565-7566, 1998.
Dickson, J. Magn. Magn. Mater., 203:46, 1999.
Gamez et al., Microchem. J., 71:193-204, 2002.
Gardea-Torresdey et al., Environ. Sci. Technol., 34:4392, 2000.
Gardea-Torresdey et al., J. Hazard. Mater, B:69:41, 1999.
Gardea-Torresdey et al., J. Nanopart. Res., 1:397, 1999.
Gardea-Torresdey et al., Nano Lett., 2:397-401, 2002.
Greene et al., EnViron. Sci. Technol., 20:627, 1986.
Jahn, J. Struct. Biol., 127:106, 1999.
Klaus et al., Proc. Natl. Acad. Sci. USA, 96:13611-13614, 1999.
Koningsberger and Prins, In: Chemical Analysis, X-ray Absorption: Principles, Applications, Techniques of EXAFS SEXAFS and XANES; John Wiley and Sons, NY, Vol. 91, 1988.
Kuyicek and Volensky, Biorecovery, 1:189, 1989.
Lytle et al., Environ. Sci. Technol., 32:3087-3093, 1998.
McInnes et al., J. Geochem. Explor., 57:227, 1996.
Montano et al., Chem. Phys. Lett., 164:126-130, 1989.
Mukherjee et al., Nano Lett., 1:515, 2001.
Naiwa, In: HandBook of Nanostructural Materials and Nanotechnology; Academic Press, NY, 1-5, 2000.
Penner-Haln, Coord. Chem. Rev., 190-192:1101-1123, 1999.
Peralta et al., Bull. Environ. Contam. Toxicol., 66:727, 2001.
Priyabrata et al., Nano Lett., 1:515-519, 2001.
Rao and Cheetham, J. Mater. Chem., 11:2887-2894, 2001.
Ravel, J. Synchrotron Radiat., 8:314-316, 2001.
Rehr and Albers, Phys. Rev. B Condens Matter, 41(12):8139-8149, 1990.
Ressler, J. Synchrotron Radiat., 5:118-122, 1998.
Ressler, J. Phys., IV:7, 1997.
Salt et al., Environ. Sci. Technol., 33:713, 1999.
Stephen and McCaughton, Curr. Opin. Biotechnol., 10:230, 1999.
Tomita et al., Ultramicroscopy, 16:9, 1985.
Urban et al., Phys. D, 28:247, 1993.
Winterbourn et al., Sci. Tot. Environ., 245:45, 2000.
Yacaman et al., J. Vac. Sci. Technol., B19:1091, 2001.

1. A method of producing a precious metal nanoparticle in a plant comprising:
(a) selecting a plant growth environment comprising a precious metal source;
(b) growing a plant in said plant growth environment; and
(c) isolating said precious metal nanoparticle.

2. The method of claim 1, wherein said precious metal is gold.

3 The method of claim 1, wherein said precious metal is silver.

4. The method of claim 1, wherein said precious metal is platinum.

5. The method of claim 1, wherein said plant is a dicot.

6. The method of claim 5, wherein said dicot is of the division Magnoliophyta.

7. The method of claim 6, wherein said dicot is alfalfa.

8. The method of claim 1, wherein isolating comprises isolating a part of said plant.

9. The method of claim 8, wherein said plant part is a leaf, a stem, or a root.

10. The method of claim 9, further comprising disrupting said plant part by physical, chemical or biological methods.

11. The method of claim 10, wherein the physical methods comprise pressing, grinding, sonication or burning.

12. The method of claim 10, wherein the chemical methods comprise digestion or extraction.

13. The method of claim 10, wherein the biological methods comprise enzymatic degradation or microbial degradation.

14. The method of claim 8, wherein isolating comprises one or more of chromatography, centrifugation or electrophoresis.

15. The method of claim 1, wherein growing comprises planting a seed, a sprout of said plant, or said plant.

16. The method of claim 1, further comprising creating said plant growth environment comprising a precious metal source.

17. The method of claim 16, wherein said plant growth environment is soil or liquid.

18. The method of claim 17, wherein creating said plant growth environment comprises seeding a solid growth medium with a precious metal.

19. The method of claim 18, wherein said solid growth medium is soil or agar.

20. The method of claim 17, wherein creating said plant growth environment comprises mixing a precious metal with a liquid.

21. The method of claim 16, wherein creating said plant growth environment comprises:
(i) selecting an desired particle size; and
(ii) adjusting the precious metal concentration to produce said desire particle size.

22. The method of claim 2, wherein said nanoparticles have one or more of the following characteristics:
(i) crystalline;
(ii) size of between about 2 nm and about 40 nm;
(iii) twinned structure;
(iv) icosahedral structure;
(v) zero valence.

23. The method of claim 3, wherein said nanoparticles have one or more of the following characteristics:
(i) crystalline;
(ii) size of between about 2 nm and 20 nm;
(iii) icosahedral structure;
(iv) dimeric, multimeric or wired;
(v) zero valence.
